(19) United States
(12) Patent Application Publication
(10) Pub. No.: US 2022/0287589 A1
(43) Pub. Date: Sep. 15, 2022

(54) Instrument, System and Methods for Use in Respiratory Exchange Ratio Measurement

(71) Applicants: Rijksuniversiteit Groningen, Groningen (NL); Academisch Ziekenhuis Groningen, Groningen (NL)

(72) Inventors: Charlotte Christina Roossien, Assen (NL); Gijsbertus Jacob Verkerke, Glimmen (NL); Michiel Felix Reneman, Peize (NL)

(21) Appl. No.: 17/632,346

(22) PCT Filed: Jul. 31, 2020
(86) PCT No.: PCT/NL2020/050492

§ 371 Date: Feb. 2, 2022

(30) Foreign Application Priority Data
Aug. 2, 2019 (EP) 19189792.5

(51) Int. Cl. A61B 5/083; A61B 5/087; A61B 5/00; A61B 5/097

(52) U.S. Cl. CPC A61B 5/0833; A61B 5/087; A61B 5/6803; A61B 5/097; A61B 5/0836

(57) ABSTRACT

The instrument has sensors for sensing oxygen and/or carbon dioxide content in exhaled air received in a receiving area in front of a mouth, an air flow rate sensor for sensing exhaled air flow rates in a flow rate sensing location and an air shield for shielding the receiving area and the flow rate sensing location from air flows from the environment. The air shield leaves a space between the air shield and the mouth of the person in open communication with the environment. The air flow rate sensor senses air flow speed in a location spaced from the exhaled air receiving area, rearward of a front end of the exhaled air receiving area and above a lower end of the exhaled air receiving area. In another embodiment a sensor for sensing ambient wind is provided.

### FIELD AND BACKGROUND OF THE INVENTION

The invention relates to an instrument according to the introductory portion of claim 1 and to a system according to the introductory portion of claim 10. The invention also relates to methods according to the introductory portions of each of claims 14 and 15. Such an instrument, such a system and such methods are known from European patent application 2 913 003.

Monitoring the energetic workload of physically active workers, such as fire fighters, chemical cleaners and construction workers allows to identify types of tasks of which the workload is too high to allow the task to be performed over longer periods of time without entailing an increase of the risk of health complaints and/or to allow the task to be performed without a decrease of body control that entails a safety hazard and/or a reduced quality of the result of the performed task. Other applications for which such instruments, systems methods can be used include monitoring patients during revalidation exercises, athletes during training and measuring performance capacity indicators of athletes, such as maximum oxygen intake.

A reliable method of measuring energetic workload over longer periods of time is measuring the respiratory exchange ratio (RER). The RER is the ratio of the produced volume of carbon dioxide (CO₂) to the consumed volume of oxygen (O₂) (i.e.: VCO₂/VO₂). During steady state low-intensity activity, the RER is generally 0.7 to 0.8 and up to about 0.88. During such activity, fatty acids constitute the primary fuel. During steady state higher intensity activity, the RER is between 0.85 and 1.0 indicating that a mix of fat and carbohydrates is being burned, the proportion of carbohydrates increasing with effort. During steady state very high intensity activity, the RER can exceed 1.0 as a result of hyperventilation and increased buffering of blood lactic acid from muscles, carbohydrate being the predominant fuel source. Thus the RER is a parameter indicating steady state effort in relation to maximum effort for a subject even though a range of values has been found as indicative of maximum effort.

The RER is measured by determining oxygen consumption and carbon dioxide production from oxygen and carbon dioxide contents in inhaled and exhaled air and volumes of breathed air. In some applications, measuring only (maximum) oxygen consumption or (maximum) carbon dioxide production is desired or sufficient.

Measurement of maximum oxygen consumption and carbon dioxide production conventionally requires a time-consuming and expensive laboratory test. It cannot be used to measure in a working situation. For measurement of the RER in working situations, wearable breathing gas analyzers are commercially available such as the ‘Oxycon Mobile’ available from Vyaire Medical, U.S., the ‘K5’ available from Cosmed, Italy and the VmaxST available from SensorMedics, U.S. A disadvantage of such systems is that its use involves wearing a facemask or a mouthpiece, which increases breathing resistance, is experienced as claustrophobic and oppressive, increases the breathing resistance and makes communication by speech practically impossible. Especially in a working situation of physically active workers communication is in most cases very important, also for safety. Additionally, it is uncomfortable, which makes it less suitable for measuring during a full working day. A presently used alternative is the use of a widely commercially available heart rate monitor. While inexpensive, unobtrusive and practical in use, the measurement properties for individual determination of energetic workload are unacceptable, unless the heart rate is individually calibrated with a previously mentioned laboratory test and other conditions apply (e.g. no heart rate medication).

From European patent application 2 913 003 an instrument for collecting a sample of humanly breathed air is known which includes a funnel or tube shaped housing element arranged for guiding inhaled and/or exhaled air along, around or through a sensor unit. The funnel or tube shaped housing element is provided with an opening so that the inhaled and/or exhaled air is at least partially guided through the opening. The funnel or tube shaped housing element may be held at a distance from the mouth, nose and/or other portions of the face, so that the funnel or tube shaped housing element provides a partially open volume of space of the inhaled and/or exhaled air. This allows avoiding heat accumulation, pressure area and/or a sense of constriction and allows comfort of wear and use to be improved.

### SUMMARY OF THE INVENTION

It is an object of the present invention to provide an instrument, a system and a method that allows accurate measurement of volumes of inhaled and exhaled air and of concentrations of oxygen and/or carbon dioxide in at least the exhaled air while leaving an at least partially open volume of space directly in front of the nose and mouth of the person.

According to the invention, this object is achieved by providing an instrument according to claim 1 and a method according to claim 15. Because the air flow rate sensor is an air flow speed sensor for sensing air speed in an area spaced from the exhaled air receiving area or areas rearward of a front end of the exhaled air receiving area or areas and above a lower end of the exhaled air receiving area or areas, the velocity of exhaled air over a period of time can be used as an accurate measure of the volume of exhaled air over that period of time. In particular, in this air flow rate sensing location, exhaled air from the mouth as well as exhaled air from the nose is still sufficiently far upstream of the exhaled air receiving area so as to be undisturbed by elements receiving the exhaled air for sensing the oxygen and/or carbon dioxide contents in the received air. This allows the speed of air exhaled from both the nose and the mouth to be measured accurately without significant disturbance by the oxygen and/or carbon dioxide sensing surfaces.

The invention can also be embodied in a system according to claim 11 and in a method according to claim 16.

Flows of air other than flows of air inhaled and exhaled by the person can disturb the measurement of the flow rate of air inhaled and/or exhaled by the person. By providing a wind speed sensor for sensing wind speed in a wind speed sensing location outside of an area rearward of the shield, flows of air other than flows of air inhaled and exhaled by the person, in particular ambient air, which influence the measurement of the flow rate of air inhaled and/or exhaled by the person, can be measured as well. The system according to this embodiment further comprises a signal processor connected to the oxygen and/or carbon dioxide sensors for receiving signals representing measured oxygen and/or carbon dioxide contents, connected to the air flow rate sensor for receiving signals representing measured air flow rates and connected to the wind sensor for receiving signals representing measured wind speed. The signal processor is arranged for calculating oxygen consumption from the signals representing oxygen contents and for calculating carbon dioxide production from the signals representing carbon dioxide contents and from air flow rates while applying a correction or suppression in accordance with a value of the wind speed signal. A suppression of measured oxygen consumption signals causes signals obtained during time intervals in a given period of time in which a strong wind has been measured to be given less than full weight or no weight in the calculation of the oxygen consumption over the given period of time. If too much wind has been sensed during the entire period of time or during a too large portion of the period of time, the signal processing device may output a signal indicating that no sufficiently reliable measurement over the given period of time was possible.

Thus, the calculated oxygen consumption can be corrected and/or suppressed in accordance with sensed wind speeds. The calculated oxygen consumption can for instance be corrected in accordance with sensed wind speeds within a first range and additionally be suppressed in response to sensed wind speeds in a range that significantly affects the accuracy of even the corrected measurement of flow rates and/or oxygen contents and/or carbon dioxide contents.

Particular elaborations and embodiments of the invention are set forth in the dependent claims.

Further optional features, effects and details of the invention appear from the detailed description and the drawings.

### BRIEF DESCRIPTION OF THE DRAWINGS

FIG. 1 is a perspective view of a head of a person wearing an example of an instrument according to the invention;

FIG. 2 is a perspective view of a head of the instrument shown in FIG. 1;

FIG. 3 is a perspective partially cut-away view of an air shield, an air flow speed sensor and a portion of an air guiding conduit and sensor carrier bracket adjacent to the air shield, all of the instrument shown in FIGS. 1 and 2;

FIG. 4 is a cross-sectional side view of the air shield and air flow speed sensor shown in FIG. 3; and

FIG. 5 is a flow chart of an example of a method according to the invention of calculating a RER from signals representative of air flow speed, wind speed and oxygen contents and carbon dioxide contents of at least exhaled air.

### DETAILED DESCRIPTION

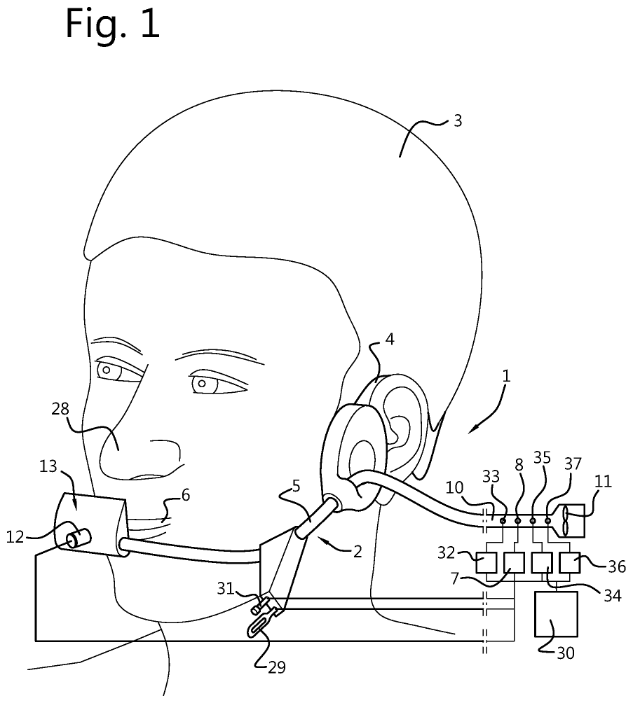

In FIGS. 1-4 an example of an instrument 1 according to the invention is shown. The instrument 1 has a bracket 2 for mounting the instrument to a head 3 of a person, of whom the RER is to be measured or monitored, in an operating position as shown in FIG. 1.

The bracket 2 is composed of a head engagement portion 4 and a sensor carrier portion 5 projecting forwardly and downwardly from the head engagement portion 4 when the instrument 1 is in the operating position. The bracket 2 is arranged such that the sensor carrier portion 5 can extend to an area closely in front of a mouth 6 of the person when the head engagement portion 4 is in engagement with the head 3 holding the instrument 1 in the operating position.

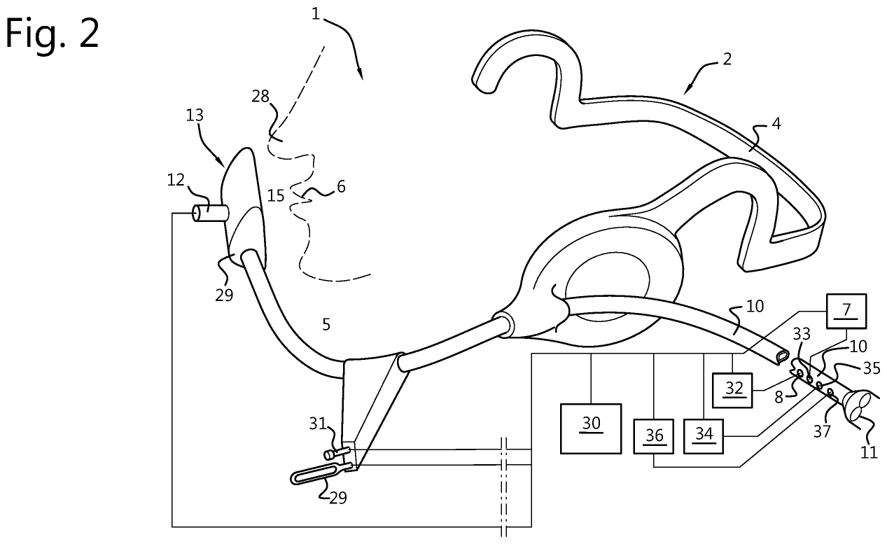

The instrument 1 further includes an oxygen content sensor 7 for sensing oxygen content in air received in exhaled air receiving areas 9 (see FIGS. 2-4) in front of the mouth 6. In this example, the oxygen sensor 7 has a sensing interface 8 in a conduit 10 from the exhaled air receiving areas 9 to a suction generator in the form of a ventilator 11. In this example, the suction generator is located downstream of the oxygen sensing interface 8, but the ventilator or other air displacement member may also be located upstream of the oxygen sensing interface. The air receiving areas 9 are formed by openings in a tube forming an upstream end of the conduit 10. A carbon dioxide sensor 36 with a carbon dioxide sensor sensing interface 37 in the conduit 10 is provided or sensing carbon dioxide contents in the air received in the exhaled air receiving area 9.

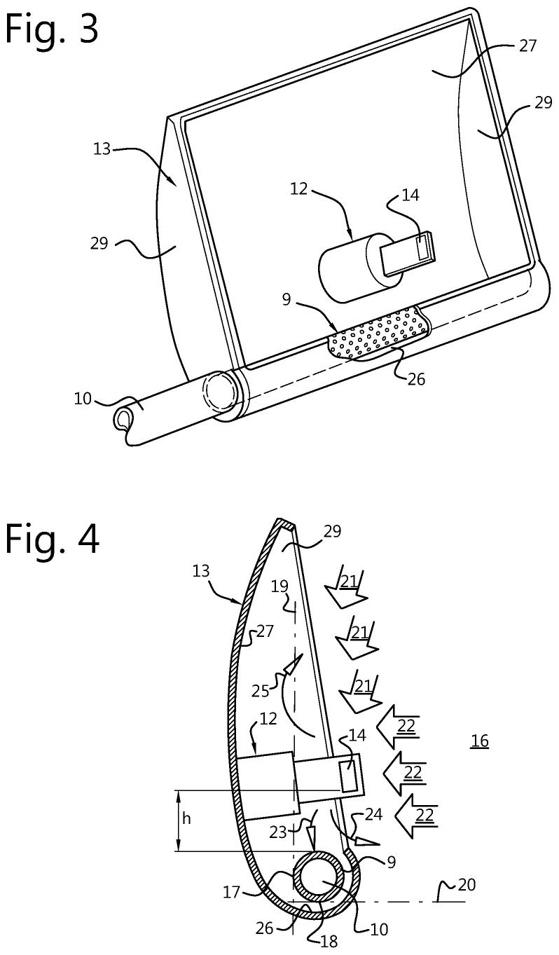

An air flow rate sensor 12 (see FIGS. 3 and 4) is arranged for sensing exhaled air flow rates in an exhaled air flow rate sensing location. In this example, the air flow rate sensor has a sensor interface 14 located in the air flow rate sensing location. However, the air flow rate sensor may also have a sensing interface located outside the air flow rate sensing location, for instance in the form of a pressure sensing interface located in a pitot tube having an open end in the air flow rate sensing location or located in a conduit communicating with a venturi in the air flow rate sensing location.

In front of the air receiving areas 9 and the air flow rate sensing location, an air shield 13 is provided for shielding the exhaled air receiving areas 9 and the air flow rate sensing location from air flows from an environment forwardly of the shield 13. The air shield 13 is shaped and positioned for leaving a space 15 (see FIG. 2) between the air shield 13 and the mouth 6 of the person, which space 15 is in open communication with the environment.

The air flow rate sensor is an air flow speed sensor 12 having a hot air surface sensing interface 14. The air flow rate sensing location is located spaced from the exhaled air receiving areas 9 in an area 16 (see FIG. 4) rearward of a front end 17 of the exhaled air receiving areas 9 and above a lower end 18 of the exhaled air receiving areas 9. In the example shown in FIG. 4, this air flow rate sensing location 16 is located to the right of vertical dash-and-dot line 19 and above horizontal dash-and-dot line 20.

The instrument 1 is wearable and can be used for sensing at least oxygen or carbon dioxide contents and flow rates of air exhaled by a human person when attached in an operating position to the head 3 of the person, as is shown in FIG. 1.

The air flow rate sensor is an air flow speed sensor 12 for sensing air speed in an area 16 outside of the mouth and nose, in front of the mouth, spaced from the exhaled air receiving areas 9, rearward of a front end 17 of the exhaled air receiving areas 9 and above a lower end 18 of the exhaled air receiving areas 9. In this air flow rate sensing location 16, exhaled air from the mouth 6, as indicated by arrows 21, or from the nose 28, as indicated by arrows 22, is still sufficiently far upstream of the exhaled air receiving areas 9 so as to be undisturbed by the tube in which the openings form the exhaled air receiving areas 9 for receiving the exhaled air are provided. This allows the speed of air exhaled from both the nose and the mouth to be measured accurately, so that the velocity of exhaled air over a period of time can be used as an accurate measure of the volume of exhaled air over that period of time.

In this example, the sensing interface 8 for sensing oxygen contents in the air received via the openings forming the exhaled air receiving areas 9 is located downstream of the exhaled air receiving areas 9. It is, however, also conceivable to arrange the sensing interface or interfaces directly in the exhaled air receiving areas. In such an embodiment, the sensing interface or interfaces form the exhaled air receiving area or areas. Also in such an embodiment, the location of the air flow rate sensing location spaced from the exhaled air receiving area or areas, rearward of a front end of the exhaled air receiving area or areas and above a lower end of the exhaled air receiving area or areas ensures that exhaled air from the mouth and/or from the nose, is still sufficiently far upstream from structures in the exhaled air receiving area or areas so as to be undisturbed, which allows the air speed to be measured accurately.

For reliably ensuring that the speed of the exhaled air flow is undisturbed where the air speed is measured, the air flow speed sensing area 16 is preferably located at a distance h of at least 5 mm and more preferably at least 10 mm above the exhaled air receiving area or areas 9.

For reliably receiving both exhaled air from the nose 28 (arrows 21) and exhaled air from the mouth 6 (arrows 22) free from ambient air, the exhaled air receiving areas 9 are located behind a lower quarter portion of the air shield 13. Thus, air from the nose can flow along the shield 13 towards the exhaled air receiving areas 9 (arrow 23 in FIG. 4) and partially past the exhaled air receiving areas 9 (arrow 24 in FIG. 4) while being shielded from mixing with ambient air (both upstream and downstream of the air flow speed sensing interface 14), while air from the mouth flows directly against the air shield 13 and drives any ambient air away from the exhaled air receiving areas 9. After having passed the air flow speed sensing interface 14 air exhaled from the mouth can flow partially to the exhaled air receiving areas 9 (arrow 23), partially past the exhaled air receiving areas 9 (arrow 24) and partially away from the exhaled air receiving areas 9 (arrow 25). Thus, a particularly small air shield 13 is sufficient for keeping ambient air away when receiving exhaled air from the nose 28 and when receiving exhaled air from the mouth 6 for measuring the contents of oxygen and/or carbon dioxide in the exhaled air.

For guiding exhaled air from the nose 28 and the mouth 6 towards the exhaled air receiving areas 9 and effectively keeping ambient air away from the exhaled air receiving areas 9, the air shield 13 has an upwardly facing wall surface 26 extending from underneath the exhaled air receiving areas 9 until rearward of the exhaled air receiving area.

The air shield 13 has a rearward facing wall surface 27 extending from forwardly of the exhaled air receiving areas 9 until upwardly of the air flow rate sensing location formed by the air flow speed sensing interface 14. This rearward facing wall surface 27 is particularly effective for, on the one hand, deflecting exhaled air 21 from the nose 28 downward along the air flow rate sensing location and deflecting exhaled air 22 from the mouth 6 upward after at least a portion of that exhaled air has passed the air flow rate sensing location, so that a particularly representative flow of air along the exhaled air receiving areas 9 is obtained, regardless whether the air is exhaled via the nose 28 of via the mouth 6.

For effectively deflecting the exhaled air while causing little flow resistance, the rearward facing wall surface 27 of the air shield 13 is curved with a hollow curvature. The curvature preferably has a radius or radii of curvature between 5 and 15 cm. The curvature preferably extends over an angle of deflection of 5 to 15°.

If, as in the present example, the upwardly facing wall surface 26 is contiguous with the rearward facing wall surface 27, exhaled air is lead to the exhaled air receiving areas 9 particularly effectively and inflow of ambient air between the upwardly facing wall surface 26 and the rearward facing wall surface 27, which could mix with the exhaled air is avoided.

For driving an air stream through the entry passages 9 into and through the air duct 10, the air displacement device 11 is provided. The oxygen content sensor 7 includes an oxygen sensing surface 8 downstream of the entry passage 9. Thus, in use at least a portion of the exhaled air that reaches the exhaled air receiving areas 9 is drawn away through the opening 9 and towards the oxygen sensing surface 8 and the carbon dioxide sensing interface 37. This allows exhaled air to be sampled in a representative manner throughout each cycle of exhaling air, because air is continuously drawn in from a flow of air along the exhaled air receiving areas 9.

While air is inhaled, ambient air will flow to the air receiving areas 9 and is drawn away through the opening 9 and towards the oxygen sensing surface 8 and the carbon dioxide sensor sensing interface 37. This allows to intermittently measure the oxygen and carbon dioxide contents of inhaled air using the same sensing interfaces 8, 37. It is also possible to measure the concentrations of oxygen and carbon dioxide of inhaled (usually ambient) air outside the air conduit 10, for instance using separate sensors exposed to ambient air or using a separate apparatus measuring concentrations of oxygen and carbon dioxide in ambient air. Since concentrations of oxygen and carbon dioxide in ambient air tend to vary quite slowly, these concentrations can be measured at a much lower frequency than the concentrations of oxygen and carbon dioxide in exhaled air.

The entry openings 9 are distributed over a major portion of a width of the air shield 13, so that exhaled air is sampled over the major portion of the width of the air shield 13, which is also advantageous for representative sampling from the flow of exhaled air. The major portion is preferably at least half of the width of the air shield, more preferably at least 75% of the width of the air shield and yet more preferably essentially the full width of the air shield 13 minus portions of the air shield occupied by end walls 29. The exhaled air receiving areas 9 are, on average, located centrally in lateral directions relative to the air shield 13, so that, on average, exhaled air is sampled from a laterally central portion of the air shield 13.

Instead of a plurality of air receiving areas 9, a single air receiving area can be provided, for instance in the form of a single, for instance elongate, opening. Instead of by an opening, the air receiving area or areas may also be formed by the sensing surface or, respectively, the sensing surfaces for sensing the contents of carbon dioxide and oxygen in the air received at this surface or at these surfaces.

The instrument 1 is further equipped with a wind speed sensor 29 for sensing wind speed in a wind speed sensing location outside of the area 15 rearward of the shield 13. The instrument 1 is part of a system for measuring oxygen consumption, which further includes a signal processor 30 connected to the oxygen sensor 7 for receiving signals representing measured oxygen contents, to the carbon dioxide sensor 36 for receiving signals representing measured carbon dioxide contents, to the air flow rate sensor 12 for receiving signals representing measured air flow rates and to the wind sensor 29 for receiving signals representing measured wind speed. The signal processor 30 is arranged for calculating oxygen consumption from the signals representing the oxygen contents and the air flow rates while applying a correction or suppression in accordance with a value of the wind speed signal. The signal processor 30 is also arranged for calculating carbon dioxide production from the signals representing the carbon dioxide contents and the air flow rates while applying the correction or suppression in accordance with the value of the wind speed signal.

Flows of air other than the flows of air inhaled and exhaled by the person can disturb the measurement of the flow rate of air inhaled and/or exhaled by the person. Such flows of for instance ambient air may in particularly be caused by wind, which may for instance be weather related wind or draft and/or wind caused by movement of the person who may for instance be walking, running, riding or be located on a moving vessel. By providing a wind speed sensor 29 for sensing wind speed in a wind speed sensing location outside of the area 15 rearward of the shield 13, flows of air other than flows of air inhaled and exhaled by the person, which influence the measurement of the flow rate of air inhaled and/or exhaled by the person, can be measured as well. The correction or suppression of the measured contents signals allows correcting or suppressing of the measured oxygen consumption and carbon dioxide production in accordance with sensed wind speeds. The calculated oxygen consumption and carbon dioxide production can for instance be corrected in accordance with sensed wind speeds within a first range, for instance to compensate for admixing of ambient air into the exhaled air, and be suppressed in response to sensed wind speeds in a range that does not allow sufficiently accurate measurement of flow rates and/or oxygen and carbon dioxide contents.

The instrument 1 is further provided with a wind direction sensor 31 for sensing a direction of the wind. The wind direction sensor 31 also communicates with the signal processor 30. The signal processor 30 may for instance be arranged for responding differently to wind from ahead than to side wind or wind from above. In response to wind from ahead of a given wind speed, a correction or suppression is preferably less than in response to wind of the same speed from a side or from above, which tends to cause more admixing of ambient air in to the exhaled air than wind from ahead.

For reliable sensing of wind from any direction (e.g. head wind, side wind, rear wind or wind from above or below), the wind speed sensor 29 is located laterally spaced from the air shield 13. The distance from the wind speed sensor 29 to the air shield 13 is preferably at least 5 cm and more preferably at least 7 cm.

For allowing a further increase in the reliability of measuring oxygen consumption, the instrument 1 is further equipped with a temperature sensor 32 with a temperature sensing interface 33 in the conduit 10 and a relative humidity sensor 34 with a humidity sensing interface 35 in the conduit 10.

Correction or suppression of the measured oxygen consumption and carbon dioxide production in accordance with air flow speeds in a location outside of the area 15 between the shield 13 and the mouth and nose of the person wearing the instrument can also be advantageously applied if the flow rate of exhaled air is measured in a different manner than using an air speed sensor for measuring air speed in areas spaced from the exhaled air receiving area and above the lower end of the exhaled air receiving area and to the rear of the front end of the exhaled air receiving area, for example by measuring the pressure of exhaled air in the exhaled air receiving area.

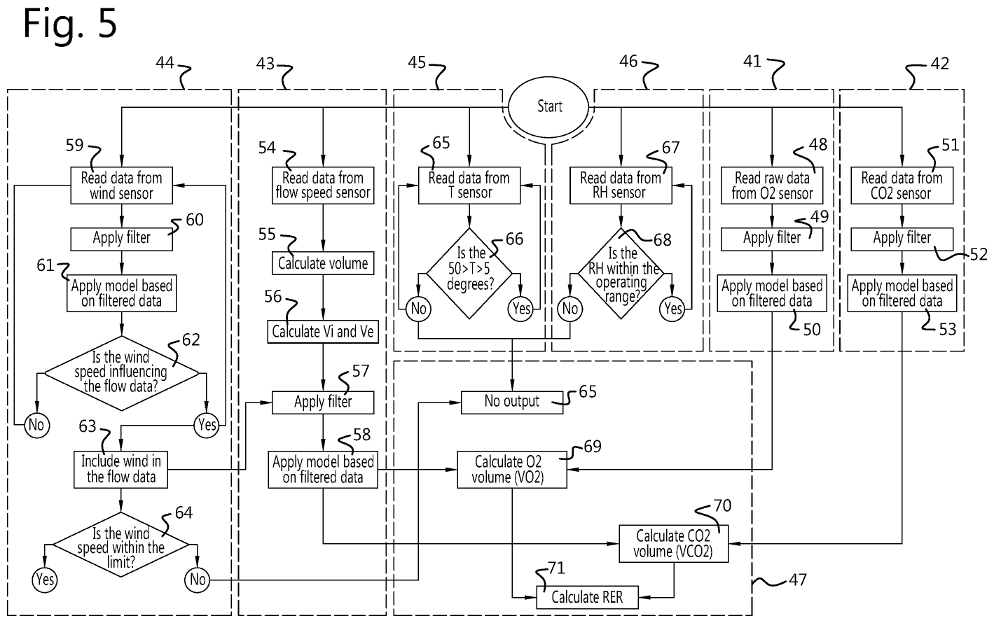

Operation of the system according to the described example is further described with reference to the flow chart shown in FIG. 5. The computer program for determining the RER from output signals of the sensors 7, 12, 32, 34 and 36 is composed of six modules. Three main modules are an oxygen contents determination module 41, a carbon dioxide contents determination module 42 and a flow rate determination module 43. Other modules are a wind condition correction module 44, temperature and relative humidity checking modules 45 and, respectively, 46, and a measurement output determination module 47.

In step 48 of oxygen contents determination module 41, raw oxygen concentration data obtained over a given period of time are read from a memory containing captured oxygen contents signals received from the oxygen contents sensor 7. Preferably, the concentration data are directly indicative of oxygen concentrations, by converting and calibrating direct sensor output signals. In step 49 a filter is applied to the read data. The filter may for instance be a Kalman filter, a low pass filter or a recursive least square (RLS) filter. In step 50 a model is applied to the filtered data to obtain to further reduce noise from the filtered data, for instance by fitting the filtered concentration data obtained over the given period of time to characteristics of variation of oxygen concentration over time during a breathing cycle.

In step 51 of carbon dioxide determination module 42, raw carbon dioxide concentration data obtained over the same period of time are read from a memory containing captured carbon dioxide contents signals received from the carbon dioxide contents sensor 36. Preferably, the concentration data are directly indicative of carbon dioxide concentrations, by converting and calibrating direct sensor output signals. In step 52 a filter is applied to the read data. The filter may for instance be a Kalman filter, a low pass filter or an RLS filter and is preferably the same filter as the filter applied to the oxygen concentration data. In step 53 a model is applied to the filtered data to obtain to further reduce noise from the filtered data, for instance by fitting the filtered concentration data obtained over the given period of time to characteristics of variation of carbon dioxide concentration over time during a breathing cycle. This model may differ from the model applied to the filtered oxygen concentration data.

In step 54 of flow rate determination module 43, raw flow speed data over the given period of time are read from a memory containing captured flow speed signals received from the flow speed sensor 12. The flow speed data preferably represent flow speed directly, so that for instance a resistance signal from a hot wire flow speed sensor has already been converted into a calibrated flow speed signal. In step 55, flow rates, i.e. volumes per unit of time, are calculated from the read flow speed data. These calculations include determining a breathing frequency from the number of peaks and/or valleys in the flow speed signal per unit of time or from the time between peaks and/or valleys and determining the volumes from the measured flow speeds and the breathing frequency.

Since the relationship between air flow speed and air flow rate tends to be different for air exhaled through the nose from air exhaled through the mouth, preferably the conversion from air flow speed to air flow rate is made in accordance with mutually different relationships for breathing out through the nose and breathing out through the mouth. Whether breathing out is carried out through the nose or through the mouth can be taken into account by including a breathing frequency from cyclic variations of the air flow speed over a period of time and air flow speeds in the determination of the volumetric breathing air flow rate. This can for instance be accomplished using the following formula:

Air flow rate (L/s)=Air Flow (m/s)*(Breathes per minute*b)−c

in which b is a factor determining the extent to which the breathing frequency affects the flow rate and c is a constant.

The values of b and c depend on the actual design of the device. The value of b may for instance be between 0.03 and 0.12 and c may for instance be between 0.3 and 1.2. In step 56 inhaled volumes Vᵢ and exhaled volumes Vₑ are calculated by integration of the calculated flow rate over several breathing cycles from the flow rates calculated in step 55. In step 57 a filter is applied to the inhaled volumes Vᵢ and exhaled volumes Vₑ. The filter may for instance be a Kalman filter, a low pass filter or an RLS filter. In step 58 a model is applied to the filtered volume data, for instance by fitting the filtered volumetric data obtained over the given period of time to a model of characteristics of the typical variation of the flow rate over time during a breathing cycle.

In step 59 of wind condition correction module 44, wind speed data are read from captured wind speed signals received from the wind speed sensor 29. The wind speed data are preferably directly indicative of wind speed, so that for instance a resistance signal from a hot wire wind speed sensor has already been converted into a calibrated wind speed signal. Wind direction data are also read from captured wind direction signals received from the wind direction sensor 31. In step 60 a filter is applied to the read data. The filter may for instance be a Kalman filter, a low pass filter or an RLS filter. In step 61, a model is applied to the filtered data to obtain a normalized value for the influence of the wind on the measured flow rate of exhaled air. In step 62, it is determined whether the wind speed at the determined wind direction is above a first critical level at which the relationship between, on the one hand, measured flow speeds of inhaled and exhaled air and, on the other hand, inhaled volumes Vᵢ and exhaled volumes Vₑ is influenced by the wind. If it is not, the wind condition correction module 44 returns to step 59 to continue monitoring wind conditions. If the wind speed at the determined wind direction is determined to be above the first critical level, the wind condition correction module 44 also returns to step 59 to continue monitoring wind conditions, but additionally continues to step 63 in which the influence of wind speed and wind direction is stored for inclusion in the air flow rate data to which filter 57 is applied. The result of step 63 is filtered in the filtering step 57 of the flow rate determination module 43 and entered into processing step 58 to correct the inhaled volumes Vᵢ and exhaled volumes Vₑ for the influence of ambient wind. For instance, up to wind speeds of about 10 m/s+/−2 m/s depending on wind direction, the influence may be determined in step 61 on the basis of wind speed, wind direction, while extent to which the filtered influence value is used for correcting the measured flow rate of exhaled air in processing step 58 depends on the value of the measured exhaled air flow rate (the higher the measured flow rate, the higher the wind speed must be for generating a given disturbance).

After step 63, the wind condition correction module 44 further continues to step 64 in which it is determined whether the wind speed at the determined wind direction is above a second critical level for that wind direction, e.g. above about 10 m/s+/−2 m/s depending on wind direction, higher than the first critical level for that wind direction, at which the relationship between, on the one hand, measured flow speeds of inhaled and exhaled air and, on the other hand, inhaled volumes Vᵢ and exhaled volumes Vₑ is disturbed by the wind to such an extent that no sufficiently reliable result can be obtained. If it is not (i.e. the wind speed at the determined direction is below the second critical limit for that direction), the wind condition correction module 44 is not further affected. If the wind speed at the determined wind direction is determined to be above the second critical level for that wind direction, the wind condition correction module 44 triggers a no output step 65 of the measurement output determination module 47. The no output step 65 signals that no reliable measurement could be made to an output and registration interface.

In step 65 of temperature checking module 45, temperature data indicating ambient temperature are read from a memory containing temperature signals received from the temperature sensor 32. In step 66, it is determined whether the temperature is within an allowed range. The end points of this range depend on the types of oxygen and carbon dioxide concentration sensors and may for instance be 5° C. and 50° C. If the temperature is within the allowable range, the temperature checking module 45 returns to step 65 to continue monitoring temperature conditions. If the temperature is determined to be outside the allowable range, the temperature checking module 45 also returns to step 65 to continue monitoring temperature conditions, but additionally causes trigger data to be outputted to the no output step 65 of the measurement output determination module 47. The trigger data trigger the no output step 65 to signal that no reliable measurement could be made to an output and registration interface.

In step 67 of relative humidity checking module 46, relative humidity data indicating ambient relative humidity are read from a memory containing relative humidity signals received from the relative humidity sensor 34. In step 68, it is determined whether the relative humidity is within an allowed range in which the oxygen and carbon dioxide concentration sensors operate reliably and accurately, for example 30-85%. If the relative humidity is within the allowable range, the relative humidity checking module 46 returns to step 67 to continue monitoring relative humidity conditions. If the relative humidity is determined to be outside the allowable range, the relative humidity checking module 46 also returns to step 67 to continue monitoring relative humidity conditions, but additionally causes trigger data to be outputted to the no output step 65 of the measurement output determination module 47. The trigger data cause the no output step 65 to output data signaling that no reliable measurement could be made to an output and registration interface.

In step 69 of the measurement output determination module 47 the volume of consumed oxygen VO₂ is determined from oxygen contents data determined in step 50 and the volume data determined in step 58. This involves integrating oxygen concentrations over inhaled and exhaled flow rates in a window of time and calculating the difference between inhaled and exhaled oxygen volumes. Unless overruled by a no output command from step 65, the determined volume of consumed oxygen VO₂ is outputted to the output and registration interface.

In step 69 of the measurement output determination module 47 the volume of produced carbon dioxide VCO₂ is determined from carbon dioxide contents data determined in step 53 and the volume data determined in step 58. This involves integrating carbon dioxide concentrations over inhaled and exhaled flow rates in a window of time and calculating the difference between inhaled and exhaled carbon dioxide volumes. Unless overruled by a no output command from step 65, the determined volume of produced carbon dioxide VCO₂ is outputted to the output and registration interface.

In step 70 of the measurement output determination module 47 the respiratory exchange ratio RER over the given window of time is determined from the volume of consumed oxygen VO₂ determined in step 69 and the volume of produced carbon dioxide VCO₂ determined in step 70 (RER=VCO₂/VO₂). Unless overruled by a no output command from step 65, the determined respiratory exchange ratio RER is outputted to the output and registration interface.

In the example, the exhaled air of which the contents of oxygen and/or carbon dioxide is to be measured is received in a plurality of air receiving areas. Essentially the same effects are also achieved when the exhaled air of which the contents of oxygen and/or carbon dioxide is to be measured is received in a single air receiving area. Furthermore, instead of or in addition to the oxygen sensor 7, a carbon dioxide sensor can be provided.

Several features have been described as part of the same or separate embodiments. However, it will be appreciated that the scope of the invention also includes embodiments having combinations of all or some of these features other than the specific combinations of features embodied in the examples.

1. A wearable instrument for sensing oxygen and/or carbon dioxide contents in and flow rates of air exhaled by a human person when attached in an operating position to a head of the person, the instrument comprising:
a bracket arranged for mounting the instrument to the head in the operating position, the bracket comprising a head engagement portion and a sensor carrier portion projecting forwardly and downwardly from the head engagement portion when the instrument is in the operating position, the bracket being arranged such that the sensor carrier portion can extend to an area closely in front of a mouth of the person when the head engagement portion is in engagement with the head holding the instrument in the operating position;
an oxygen content sensor for sensing oxygen content in air received in at least one exhaled air receiving area in front of the mouth;
a carbon dioxide content sensor for sensing carbon dioxide content in the air received in the at least one exhaled air receiving area; and
an air flow rate sensor for sensing exhaled air flow rates in an air flow rate sensing location;
an air shield in front of the air receiving area and the air flow rate sensing location for shielding the exhaled air receiving area and the air flow rate sensing location from air flows from an environment forwardly of the shield, the air shield being shaped and positioned for leaving a space between the air shield and the mouth of the person in open communication with the environment;
wherein the air flow rate sensor is an air flow speed sensor and the air flow rate sensing location is located spaced from the exhaled air receiving area, rearward of a front end of the exhaled air receiving area and above a lower end of the exhaled air receiving area.

2. The instrument according to claim 1, wherein the air flow rate sensing location is located at least 5 mm above the exhaled air receiving area.

3. The instrument according to claim 1, wherein the exhaled air receiving area is located behind a lower quarter portion of the air shield.

4. The instrument according to claim 3, wherein the air shield has an upwardly facing wall surface extending from underneath the exhaled air receiving area until rearward of the exhaled air receiving area.

5. The instrument according to claim 3, wherein the air shield has a rearward facing wall surface extending from forwardly of the exhaled air receiving area until upwardly of the air flow rate sensing location.

6. The instrument according to claim 5, wherein, seen in cross-sectional side view, the rearward facing wall surface of the air shield is curved with a hollow curvature.

7. The instrument according to claim 1, wherein the air shield has an upwardly facing wall surface extending from underneath the exhaled air receiving area until rearward of the exhaled air receiving area, and wherein the air shield has a rearward facing wall surface extending from forwardly of the exhaled air receiving area until upwardly of the air flow rate sensing location, and wherein the upwardly facing wall surface is contiguous with the rearward facing wall surface.

8. The instrument according to claim 1, wherein the exhaled air receiving area is formed by at least one opening forming an entry passage into an air duct, wherein an air displacement device is provided for driving an air stream through said entry passage into and through said air duct, and wherein the oxygen and/or carbon dioxide content sensors include sensing surfaces downstream of said entry passage.

9. The instrument according to claim 8, wherein a plurality of said entry openings is distributed over at least half of a width of said air shield or wherein said entry opening extends over at least half of the width of said air shield.

10. A system comprising the instrument according to claim 1 and a signal processor connected to the air flow speed sensor for receiving signals representing measured air flow speeds, wherein the signal processor is arranged for registering air flow speeds measured over a period of time, determining breathing frequencies from cyclic variations of the air flow speed over said period of time, and determining an air flow rate over said period of time from the breathing frequencies and the air flow speeds of exhaled over said period of time.

11. A system for measuring oxygen consumption and/or carbon dioxide production comprising a wearable instrument for sensing oxygen and/or carbon dioxide contents in and flow rates of air exhaled by a human person when attached in an operating position to a head of the person, the instrument comprising:
a bracket arranged for mounting the instrument to the head in the operating position, the bracket comprising a head engagement portion and a sensor carrier portion projecting forwardly and downwardly from the head engagement portion when the instrument is in the operating position, the bracket being arranged such that the sensor carrier portion can extend to an area closely in front of a mouth of the person when the head engagement portion is in engagement with the head holding the instrument in the operating position;
an oxygen content sensor for sensing oxygen content in air received in at least one exhaled air receiving area in front of the mouth;
a carbon dioxide content sensor for sensing carbon dioxide content in the air received in the at least one exhaled air receiving area; and
an air flow rate sensor for sensing exhaled air flow rates in an air flow rate sensing location;
an air shield in front of the air receiving area and the air flow rate sensing location for shielding the exhaled air receiving area and the air flow rate sensing location from air flows from an environment forwardly of the shield, the air shield being shaped and positioned for leaving a space between the air shield and the mouth of the person in open communication with the environment;
wherein the instrument further comprises a wind speed sensor for sensing wind speed in a wind speed sensing location outside of an area rearward of the shield; and
the system further comprises a signal processor connected to the oxygen sensor and the carbon dioxide sensor for receiving signals representing measured oxygen and/or carbon dioxide contents, to the air flow rate sensor for receiving signals representing measured air flow rates and to the wind sensor for receiving signals representing measured wind speed, the signal processor being arranged for calculating oxygen consumption from the signals representing the oxygen contents, carbon dioxide production from the signals representing the carbon dioxide contents and air flow rates while applying a correction or suppression in accordance with a value of the wind speed signal.

12. The system according to claim 11, further comprising a wind direction sensor for sensing a direction of said wind.

13. The system according to claim 11, wherein said wind speed sensor is located laterally spaced from said air shield.

14. The system according to any of the claim 11, wherein said wind speed sensor is located at least 5 cm away from said air shield.

15. A method for sensing oxygen and/or carbon dioxide contents in and flow rates of air exhaled by a human person using an instrument attached in an operating position to a head of the person, the instrument comprising:
an oxygen content sensor sensing oxygen content in air received in at least one exhaled air receiving area in front of a mouth of the person;
a carbon dioxide content sensor sensing carbon dioxide content in the air received in the at least one exhaled air receiving area; and
an air flow rate sensor sensing exhaled air flow rates in an air flow rate sensing location;
an air shield in front of the air receiving area and the air flow rate sensing location shielding the exhaled air receiving area and the air flow rate sensing location from air flows from an environment forwardly of the shield, the air shield leaving a space between the air shield and the mouth of the person in open communication with the environment;
wherein the air flow rate sensor senses air flow speed sensor and the air flow rate sensing location is located spaced from the exhaled air receiving area in an area rearward of a front end of the exhaled air receiving area and above a lower end of the exhaled air receiving area.

16. A method for measuring oxygen consumption and/or carbon dioxide production using a system comprising a wearable instrument sensing oxygen and/or carbon dioxide contents in and flow rates of air exhaled by a human person, the instrument being attached in an operating position to a head of the person, the instrument comprising:
an oxygen content sensor sensing oxygen content in air received in at least one exhaled air receiving area in front of the mouth;
a carbon dioxide content sensor sensing carbon dioxide content in the air received in the at least one exhaled air receiving area; and
an air flow rate sensor sensing exhaled air flow rates in an air flow rate sensing location;
an air shield in front of the air receiving area and the air flow rate sensing location shielding the exhaled air receiving area and the air flow rate sensing location from air flows from an environment forwardly of the shield, the air shield leaving a space between the air shield and the mouth of the person in open communication with the environment;
wherein the instrument further comprises a wind speed sensor sensing wind speed in a wind speed sensing location outside of an area rearward of the shield; and
the system further comprises a signal processor receiving signals representing measured contents of oxygen and/or carbon dioxide, signals representing measured air flow rates and signals representing measured wind speed, the signal processor calculating oxygen consumption from the signals representing the contents of oxygen, carbon dioxide production from the signals representing the contents of carbon dioxide and air flow rates while applying a correction or suppression in accordance with a value of the wind speed signal.
